(19) United States
(12) Patent Application Publication
(10) Pub. No.: US 2017/0360743 A1
(43) Pub. Date: Dec. 21, 2017

(54) BIFLAVONE COMPOUND AND USES THEREOF FOR TREATING CANCERS AND PREPARING DRUGS

(71) Applicant: FUJIAN MEDICAL UNIVERSITY, Fuzhou City, Fujian (CN)

(72) Inventors: Xinhua LIN, Fuzhou City (CN); Hong YAO, Fuzhou City (CN); Shaoguang LI, Fuzhou City (CN); Youjia WU, Fuzhou City (CN); Yuxia SUI, Fuzhou City (CN)

(21) Appl. No.: 15/539,613

(22) PCT Filed: May 4, 2016
(86) PCT No.: PCT/CN2016/080931

§ 371 Date: Jun. 23, 2017

(30) Foreign Application Priority Data
Dec. 3, 2015 (CN) 201510882246.0

(51) Int. Cl. A61K 31/352; A61K 9/00

(52) U.S. Cl. CPC A61K 31/352; A61K 9/0019

(57) ABSTRACT

Provided in the present invention is a compound having these structure of formula I or a pharmaceutically acceptable salt thereof. Also provided in the present invention is a pharmaceutical composition containing the compound, and a use of the compound for treating cancers.

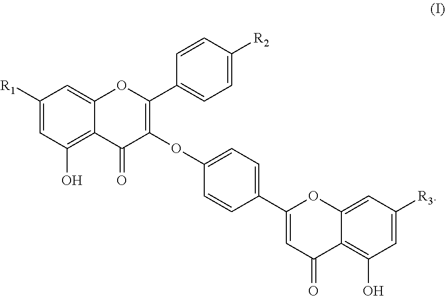

The present application claims priority of the Chinese patent application numbered 201510882246.0 entitled Biflavonoid compounds and use thereof for treating cancers and manufacturing medicaments, filed on Dec. 3, 2015. The disclosure of this application is incorporated by reference in its entirety.

### FIELD OF THE INVENTION

The present invention relates to biflavonoid compounds, pharmaceutical composition and use for treating cancers thereof. Particularly, the present invention provides biflavonoid compounds extracted and prepared from Shishangbai, and the pharmaceutical composition and use for treating cancers thereof.

### BACKGROUND OF THE INVENTION

Shishangbai, a Chinese traditional herb, is the dry whole plant of Selaginella doederleinii Hieron. Modern pharmaceutical studies have found that S. doederleinii or compositions using it as medical components are able to enhance the metabolism or functions of the reticuloendothelial system of cancer patients. Studies are carried out on the active ingredient of S. doederleinii. It has been reported that S. doederleinii comprises alkaloids, phytosterols and saponins, such as hordenine-O-α-L-rhamnopyranoside and N-methyltyramine-O-α-L-rhamnopyranoside. In addition, it is found that S. doederleinii comprises biflavones such as amentoflavone, heveaflavone and 7, 4′, 7″, 4′″-tetra-O-methyl amentoflavone.

CN104523740A, titled ‘Method for preparing Selaginella doederleinii Hieron polysaccharide extract product and application thereof’, discloses a method for preparing a Selaginella doederleinii Hieron polysaccharide extract product and the application thereof. The Selaginella doederleinii Hieron polysaccharide extract product is prepared by the steps of degreasing, extraction, alcohol precipitation, protein removal, monosaccharide and oligosaccharides removal, gel column purification and the like. The polysaccharide content in the polysaccharide extract product is more than 90%. The polysaccharide extract product has obvious anti-oxidation activity, and can be used for preparing in-vitro antitumor medicine or pharmaceutical composition.

CN104546952A, titled ‘Active component of s Selaginella doederleinii Hieron as well as preparation method and use thereof’, discloses an active component of Selaginella doederleinii Hieron as well as a preparation method and a use thereof. The method comprises the following steps: extracting and concentrating degreased residues of Selaginella doederleinii Hieron by using an ethanol solution to obtain Selaginella doederleinii Hieron liquid extract; and dissolving the Selaginella doederleinii Hieron liquid extract in water, filtering the solution by using macroreticular resin, gradient eluting the solution by using different polar solutions, collecting to obtain different polar eluents of Selaginella doederleinii Hieron, concentrating and carrying out tumor cells in vitro screening to obtain an antitumor active component. The active constituent enriches more than 70% of flavonoid components of the Selaginella doederleinii Hieron, and the active component of the Selaginella doederleinii Hieron can be prepared into a variety of dosage forms by pharmaceutical methods. The active component of the Selaginella doederleinii Hieron prepared from the preparation method is provided to have an obvious inhibitory effect on such tumor cell lines as lung cancer, leukemia, colon cancer, nasopharyngeal carcinoma and the like in vitro. The active component prepared from the preparation method can be applied to preparation of antitumor drugs or pharmaceutical compositions.

However, the known studies have not disclosed specific active ingredient in the anti-tumor compositions, therefore, the studies on active ingredient or active ingredients contained in Selaginella doederleinii are still lack of a definite material base. The manufacturing of a Selaginella doederleinii pharmaceutical product is hard to achieve good quality control. There is no report on preparing high concentration of active ingredient or active ingredients contained in Selaginella doederleinii Hieron. There is also no report on the use of active ingredient or active ingredients contained in Selaginella doederleinii Hieron for treating diseases such as cancer.

### SUMMARY OF THE INVENTION

The present invention for the first time provides the concrete active compound in the anti-tumor constituents in Selaginella doederleinii Hieron, as well as the use thereof for the treatment of cancer or for the manufacturing a medicament for treatment of cancer.

In one aspect of the present invention, the present invention provides a compound of Formula I or the pharmaceutically acceptable salt thereof:

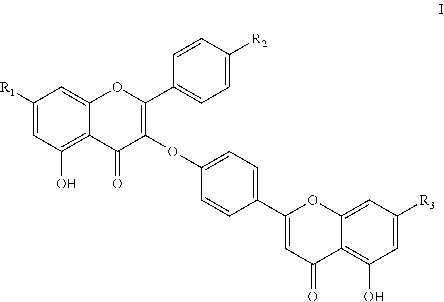

wherein

R₁, R₁ and R₃ is independently selected from H, hydroxy, lower alkyl, lower alkenyl, lower alkoxy, halo, amino, hydroxyalkyl, aminoalkyl, nitro, aryl and heteroaryl.

In one aspect of the present invention, the compound of the present invention is:

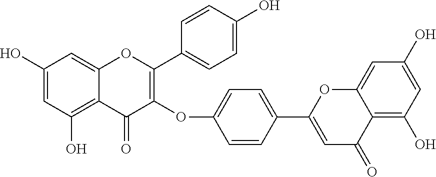 [SMILES: O=c1cc(-c2ccc(Oc3c(-c4ccc(O)cc4)oc4cc(O)cc(O)c4c3=O)cc2)oc2cc(O)cc(O)c12]

### 3-(4-(5,7-dihydroxy-4-oxo-4H-benzopyran-2-yl) phenoxyl)-5,7-dihydroxy-2-(4-hydroxyphenyl)-4H-benzopyran-4-one

In one aspect of the present invention, the present invention provides a pharmaceutical composition, which comprises a compound of formula I or the pharmaceutically acceptable salt thereof as described above and a pharmaceutically acceptable carrier, such as an aqueous carrier. In a further aspect of the present invention, the pharmaceutical composition is for treating cancer.

In one aspect of the present invention, the present invention provides a method for treating cancer, which comprises administering to a subject in need thereof a treatment effective amount of a compound of formula I or the pharmaceutically acceptable salt thereof as described above.

In one aspect of the present invention, the present invention provides use of a compound of formula I or the pharmaceutically acceptable salt thereof as described above in the manufacturing a medicament for treating cancer.

The cancers aforementioned include but are not limited to skin cancer, lung cancer, Kaposi's sarcoma, testicular cancer, lymphoma, leukemia, esophageal cancer, stomach cancer, colon cancer, breast cancer, endometrial cancer, ovarian cancer, central nervous system cancer, liver cancer and prostate cancer. Preferably, said cancer is lung cancer (such as non-small cell lung cancer), stomach cancer, esophageal cancer or prostate cancer.

### DETAILED DESCRIPTION OF THE INVENTION

The present invention will now be described more fully hereinafter with reference to the accompanying figures, which further illustrate the invention described herein. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention.

The term “alkyl” or “lower alkyl” as used herein refers to C₁ to C₈ alkyl, such as C₁ to C₃ alkyl, which may be linear or branched and saturated or unsaturated.

“Alkenyl” or “lower alkenyl” as used herein likewise refers to C₂ to C₈ alkenyl, such as C₂ to C₈ alkenyl.

“Alkoxy” as used herein refers to linear or branched, saturated or unsaturated oxo-hydrocarbon chains, containing a C₁ to C₈ alkyl, such as C₁ to C₃ alkyl, including for example methoxy, ethoxy, propoxy, isopropoxy, butoxy, and t-butoxy.

“Halo” as used herein refers to any halogen group, such as chloro, fiuoro, bromo, or iodo.

The term “hydroxyalkyl” as used herein refers to C₁ to C₄ linear or branched hydroxy-substituted alkyl, i.e., —CH₂OH, —(CH₂)₂OH, etc.

The term “aminoalkyl” as used herein refers to C₁ to C₄ linear or branched amino-substituted alkyl, wherein the term “amino” refers to the group NR′R″, wherein R′ and R″ are independently selected from H or alkyl as defined above, i.e., —NH₂, —NHCH₃, —N(CH₃)₂, etc.

The term “aryl” as used herein refers to C₃ to C₁₀ cyclic aromatic groups such as phenyl, naphthyl, and the like, and includes substituted aryl groups such as tolyl.

The term “heteroaryl” used herein refers to a 5- or 6-membered aromatic ring which includes 1, 2, 3 or 4 hetero atoms such as nitrogen, oxygen or sulfur, and such rings fused to an aryl, cycloalkyl, heteroaryl or cycloheteroalkyl ring (e.g., to provide a C₁-C₁₃ heteroaryl). Examples include but are not limited to pyridyl, pyrazolyl, thiophenyl, and the like. The heteroaryl groups may be unsubstituted or substituted with optionally include 1 to 4 substituents such as independently selected substituents from those other than “heteroaryl” identified with respect to the group R₁ herein.

“Treat” as used herein refers to any type of treatment that imparts a benefit to a patient afflicted with a disease, including improvement in the condition of the patient (e.g., in one or more symptoms), delay in the progression of the disease, prevention or delay of the onset of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

The present invention is mainly about the treatment of human subjects, but may also be employed for the treatment of other animal subjects (i.e., mammals, avians) for veterinary purposes. Mammals (including but not limited to dogs, cats, rabbits, horses, etc.) are preferred, with humans being particularly preferred.

As mentioned above, the present invention provides a compound of Formula I or the pharmaceutically acceptable salt thereof:

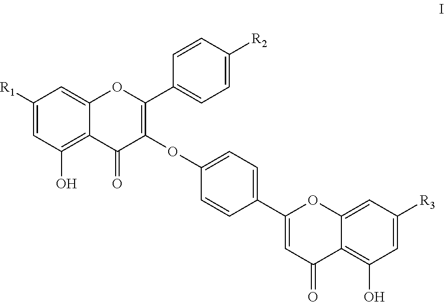

wherein

R₁, R₁ and R₃ is independently selected from H, hydroxy, lower alkyl, lower alkenyl, lower alkoxy, halo, amino, hydroxyalkyl, aminoalkyl, nitro, aryl and heteroaryl.

More specifically, one aspect of the present invention provides the compound:

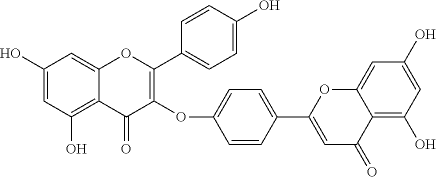 [SMILES: O=c1cc(-c2ccc(Oc3c(-c4ccc(O)cc4)oc4cc(O)cc(O)c4c3=O)cc2)oc2cc(O)cc(O)c12]

Active compounds of the present invention may be produced by the procedures described herein, or variations thereof which will be apparent to those skilled in the art.

The present invention provides a pharmaceutical composition, which comprises a compound of formula I or the pharmaceutically acceptable salt thereof as described above and a pharmaceutically acceptable carrier, wherein the compound of formula I or the pharmaceutically acceptable salt thereof is the active agent of the pharmaceutical composition.

The term “active agent” as used herein, includes the pharmaceutically acceptable salts of the compound. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

Active agents used to prepare compositions for the present invention may alternatively be in the form of a pharmaceutically acceptable free base of active agent. Because the free base of the compound is less soluble than the salt, free base compositions are employed to provide more sustained release of active agent to the target area. Active agent present in the target area which has not gone into solution is not available to induce a physiological response, but serves as a depot of bioavailable drug which gradually goes into solution.

The compounds of the present invention are useful as pharmaceutically active agents and may be utilized in bulk form. More preferably, however, these compounds are formulated into pharmaceutical formulations for administration. Any of a number of suitable pharmaceutical formulations may be utilized as a vehicle for the administration of the compounds of the present invention.

The compounds of the present invention may be formulated for administration for the treatment of a variety of conditions. In the manufacture of a pharmaceutical formulation according to the invention, the compounds of the present invention and the physiologically acceptable salts thereof, or the acid derivatives of either (hereinafter referred to as the “active compound”) are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.5% to 95% by weight of the active compound. One or more of each of the active compounds may be incorporated in the formulations of the invention, which may be prepared by any of the well-known techniques of pharmacy consisting essentially of admixing the components, optionally including one or more accessory ingredients.

The formulations of the invention include those suitable for oral, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3:318 (1986)) and typically take the form of an optionally buffered aqueous solution of the active compound. Suitable formulations comprise citrate or bis\tris buffer (pH 6) or ethanol/water and contain from 0.01 to 0.2M active ingredient.

In addition to the compounds of the formulas described herein, the present invention also provides useful therapeutic methods. For example, the present invention provides a method of inducing cytotoxicity against tumor cells, or treating a cancer or tumor in a subject in need thereof.

Subjects which may be treated using the methods of the present invention are typically human subjects although the methods of the present invention may be useful for veterinary purposes with other subjects, particularly mammalian subjects including, but not limited to, horses, cows, dogs, rabbits, fowl, sheep, and the like. As noted above, the present invention provides pharmaceutical formulations comprising the compounds of formulae described herein, or pharmaceutically acceptable salts thereof, in pharmaceutically acceptable carriers for any suitable route of administration, including but not limited to oral, rectal, topical, buccal, parenteral, intramuscular, intradermal, intravenous, and transdermal administration.

The therapeutically effective dosage of any specific compound will vary somewhat from compound to compound, patient to patient, and will depend upon the condition of the patient and the route of delivery.

The present invention also provide a method for preparing the compound of formula I of the present invention, which include the following steps: Shishangbai, the dry whole plant of Selaginella doederleinii Hieron is subjected to extraction under reflux with 70% ethanol to obtain an ethanol extraction crude material; the ethanol extraction crude material is then resuspended with water, followed by successive extraction with petroleum ether, then dichloromethane, and finally ethyl acetate to obtain an ethyl acetate extraction crude material; the ethyl acetate extraction crude material is then subjected to HPLC extraction to obtain the compound of formula I.

In one aspect of the present invention, it is provided a method for preparing the following compound:

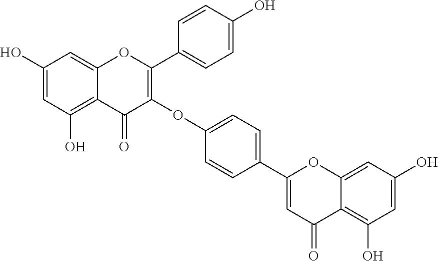 [SMILES: O=c1cc(-c2ccc(Oc3c(-c4ccc(O)cc4)oc4cc(O)cc(O)c4c3=O)cc2)oc2cc(O)cc(O)c12]

which include the following steps: Shishangbai, the dry whole plant of Selaginella doederleinii Hieron is subjected to extraction under reflux with 70% ethanol to obtain an ethanol extraction crude material; the ethanol extraction crude material is then resuspended with water, followed by successive extraction with petroleum ether, then dichloromethane, and finally ethyl acetate to obtain an ethyl acetate extraction crude material; the ethyl acetate extraction crude material is then subjected to HPLC extraction to obtain the compound of formula I.

### BRIEF DESCRIPTION OF THE DRAWINGS

FIG. 1 illustrates HPLC chromatogram of preparation of the compound of the present invention.

FIG. 2 illustrates Analysis HPLC chromatograms for purity analysis of compound of the present invention. FIG. 2(A) is the chromatogram for the 70% ethanol extraction crude material; FIG. 2(B) is the chromatogram for the ethyl acetate extraction crude material; FIG. 2(C) is an diagram showing the overlapping chromatograms for each of the separated compounds: 1(I), 2(II), 3(III), 4(IV), 5.

FIG. 3 shows photos illustrating the change of the size of tumor volume in nude mice under continuous administration in the in vivo anti-tumor activity evaluation of compound of the present invention on animal model. Animal groups: high dosage 30 mg/kg, middle dosage 15 mg/kg, low dosage 5 mg/kg; positive control group 2 mg/kg Doxorubicin.

FIG. 4 illustrates the change of the size of tumor volume in nude mice under continuous administration in the in vivo anti-tumor activity evaluation of compound of the present invention on animal model. Animal groups: high dosage 30 mg/kg, middle dosage 15 mg/kg, low dosage 5 mg/kg; positive control group 2 mg/kg Doxorubicin.

### DETAILED DESCRIPTION OF THE INVENTION

The present invention is explained in greater detail on its contents and advantages in the following non-limiting examples.

### Example 1 Preparation of Compound of Formula I

1. Handling of Shishangbai Sample

Shishangbai, the dry whole plant of Selaginella doederleinii Hieron was cut into small pieces of the length of smaller than 5 mm and then extracted under reflux with 70% ethanol. The extracted solution was then concentrated by rotary evaporation under a low temperature (40-50° C.) to give yield to an ethanol extraction crude material. The ethanol extraction crude material was then resuspended with 10× volume double stilled water, followed by successive extraction with petroleum ether, then dichloromethane, and finally ethyl acetate. The extracted solution was then concentrated by rotary evaporation under a low temperature to give yield to an ethyl acetate extraction crude material. The obtained ethyl acetate extraction crude material is then used for preparing the compound of the present invention as mentioned below.

2. Preparation by HPLC Separation

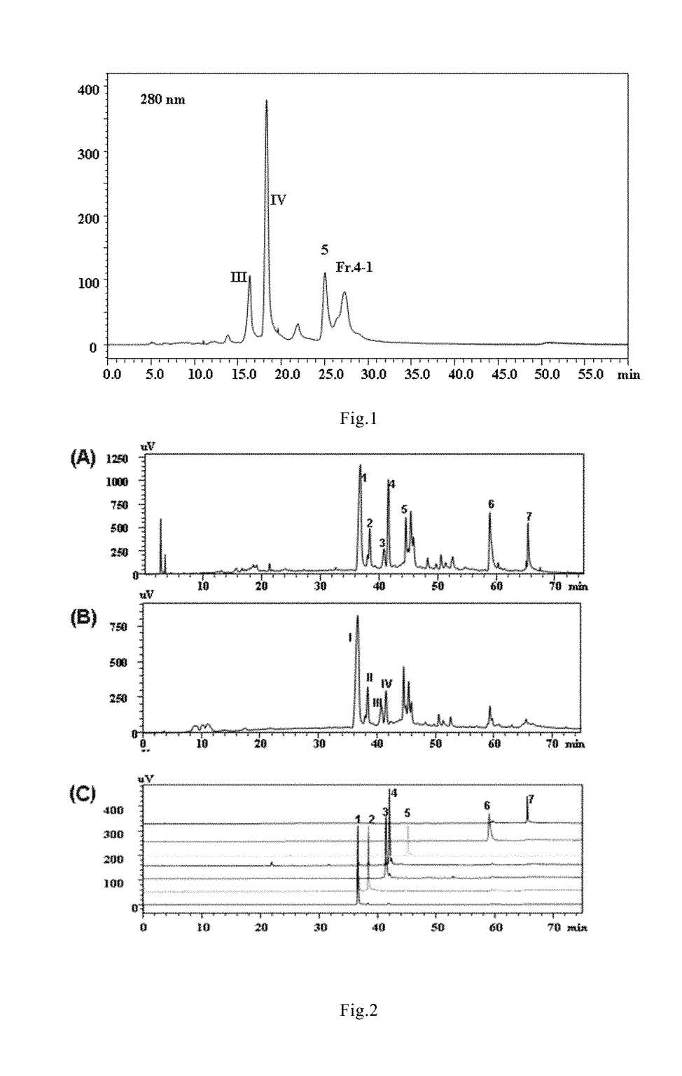

The HPLC separation was carried out with an Agilent SB-C18 column (250 mm×21.2 mm, 7 μm). The mobile phase was comprised of acetonitrile-water (44:56, v/v). The flow rate was 7 mL/min and the detection wavelength was set at 270 nm. The HPLC was carried out at RT. The injection volume was 100 μL. After the HPLC separation, as shown in FIG. 1 (preparation HPLC chromatogram), Compound III, Compound IV, Compound 5 and fraction Fr.4-1 were obtained.

Compound 5 was a powder with the color of pale yellow.

3. Purity Analysis of Compound 5 by HPLC.

FIG. 2 provides the analysis HPLC chromatograms for purity analysis of compound of the present invention. FIG. 2(A) is the chromatogram for the ethanol extraction crude material; FIG. 2(B) is the chromatogram for the ethyl acetate extraction crude material; FIG. 2(C) is a diagram showing the overlapping of the chromatograms for each of the seven separated compounds.

The HPLC settings included: A Shimadzu LC-20A HPLC system was used for the chromatographic analysis. All separations were carried out on an Ultimate XP-C18 column (Welch Materials Inc.; 4.6 mm×250 mm, 5.0 μm). The detection wavelength was set at 203 nm and 254 nm. The injection volume was 10 μL, the flow rate was 1 ml/min and column temperature is 30° C. The solvent system for the HPLC is acetonitrile-water, using a gradient elution of 10-42% (v/v), 0-30 min; 42-60% (v/v), 30-60 min; 100% (v/v), 60-70 min. The re-equilibration time was 10 min.

As calculated by the peak area normalization method, the purity of Compound 5 is higher than 95%.

Structural Identification of Compound 5

Compound 5 was subjected to thin-layer chromatography (TLC), a single round point was observed. Compound 5 was subjected to HPLC analysis using different elution system and all showed a single peak. Compound 5 was subjected to different types of spectra analysis (UV, IR, NMR, MS), the results were as shown below.

MS: (−) ESI-MS 537.2 EMHf, which means MW=538; the deduced formula is C₃₀H₁₈O₁₀; Ω=22;

UV λₘᵢₙ^{ET}: 210 nm, 268 nm, 330 nm, which are characteristic absorption peak of flavonoids;

IR λₘₐₓ^{KBr} (cm⁻¹): 3588 (OH in an aromatic ring); 3423 (C—H in an aromatic ring); 1654 (OH in an aromatic ring); 1610 (C═C in an aromatic ring); 1500 (OH in an aromatic ring); 1289 (C—O); 1028 (C—O); 838 (aryl para substitution).

1H NMR showed there were 13 Hs in the aromatic area; 13C NMR showed there were 2 carbonyl carbons (δ82.3, 6176.5) and 28 aromatic carbons, which implied that the compound is a biflavonoid. 1H NMR showed there were 2 hydroxy H (1H, s) and 12.24 (1H, s). Deduced according to the coupling constant, 8.01 (2H, d, J=8.4 Hz), 7.86 (2H, d, J=8.8 Hz), 7.26 (2H, d, J=8.8 Hz) and 6.91 (2H, d, J=8.8 Hz) constructed two AA′BB′ systems; 6.56 (1H, s, J=1.2 Hz), 6.50 (1H, s, J=1.2 Hz), 6.27 (1H, s, J=1.6 Hz) and 5.88 (1H, s, J=1.6 Hz) constructed two meta coupling H, the rest was a single H 6.85 (1H, s). 13C NMR showed δ72.9, δ99.4, δ94.7 and δ99.4, which showed C6 and C8 on the two flavone mother ring A are unsubstituted.

Based on the 1H NMR and 13C NMR data (as given in Table 1), Compound 5 was identified to have the following structure:

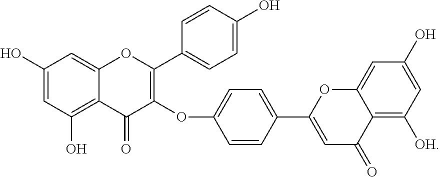 [SMILES: O=c1cc(-c2ccc(Oc3c(-c4ccc(O)cc4)oc4cc(O)cc(O)c4c3=O)cc2)oc2cc(O)cc(O)c12]

TABLE 1
NMR data of Compound 5 (DMSO-d6, 400 MHz)

| position | δC | δH | |
|---|---|---|---|
| 2 | 161.1 | | |
| 3 | 72.9 | | |
| 4 | 176.5 | | |
| 5 | 161.9 | 12.86 | (s, —OH) |
| 6 | 99.4 | 6.27 | (d, J = 1.6 Hz) |
| 7 | 165.1 | | |
| 8 | 94.6 | 6.56 | (d, J = 1.2 Hz) |
| 9 | 157.3 | | |
| 10 | 104.6 | | |
| 1′ | 120.2 | | |
| 2′, 6′ | 129.0 | 7.86 | (d, J = 8.8 Hz) |
| 3′, 5′ | 116.3 | 6.91 | (d, J = 8.8 Hz) |
| 4′ | 157.6 | | |
| 2″ | 163.5 | | |
| 3″ | 104.3 | 6.85 | (s) |
| 4″ | 182.3 | | |
| 5″ | 161.6 | 12.24 | (s, —OH) |
| 6″ | 99.4 | 5.88 | (d, J = 1.6 Hz) |
| 7″ | 164.8 | | |
| 8″ | 94.7 | 6.50 | (d, J = 1.2 Hz) |
| 9″ | 157.9 | | |
| 10″ | 104.6 | | |
| 1′″ | 125.4 | | |
| 2′″, 6′″ | 130.7 | 8.01 | (d, J = 8.4 Hz) |
| 3′″, 5′″ | 116.2 | 7.26 | (d, J = 8.8 Hz) |
| 4′″ | 160.0 | | |

### Example 2 In Vitro Anti-Tumor Activity Evaluation of Compound 5

Compound 5 obtained in Example 1 was subjected to the in vitro anti-tumor activity evaluation.

1. Preparing test solutions: taking a certain amount of compounds 5, using dimethyl sulfoxide (DMSO) to dissolve the compound, and using cell growth medium for each corresponding cell line to dilute the solution to 5 concentrations of test solutions: 500, 250, 125, 62.5, 31.25 μg/mL. The positive control doxorubicin were diluted with cell growth medium for each corresponding cell line to 3 concentrations: 5, 2.5, 1.25 μM.

2. Cell lines and cell culture

Seven human cancer cell lines, including five adhesive cancer cell lines: human gastric cancer cell line (MKN-45), non-small cell lung cancer cell line (A549), nasopharyngeal carcinoma cell line (CNE1, CNE2), large cell lung cancer cell line (PC-9); and two suspensive cancer cell lines: human promyelocytic leukemia cell line (HL60), human chronic myelogenous leukemia cell line (K562). All seven human cancer cell lines are purchased from Shanghai Chinese Academy of Sciences Cell Bank and stored in School of Medicine of Fujian Medical University.

The aforementioned seven cancer cell lines were recovered from the liquid nitrogen, and then cultured in 25 cm² flasks under the corresponding culture conditions of each cell lines (see Table 2). When the cells were grown 6-8 times of passages and the cell coverage rate in the bottom was about 80%, i.e., the cells were in the logarithmic growth phase, the cells were digested with 0.25% trypsin solution and used for passage. According to various types of cell growth rate, cell suspension were adjusted to the appropriate concentration and inoculated in 96 well plates.

TABLE 2
Seven cancer cell lines and the culturing conditions.

| Cancer | Full name of | Grow | Culture |
|---|---|---|---|
| cell lines | the cell lines | status | condition |
| K562 | human chronic myelogenous | suspensive | 5% FBS-RPMI1640, 5% CO₂, 37□ |
| | leukemia cell line | | |
| HL-60 | human promyelocytic leukemia cell | suspensive | 8% FBS-RPMI1640, 5% CO₂, 37□ |
| | line | | |
| CNE1 | Human highly differentiated | adhesive | 10% CS-RPMI1640, 5% CO₂, 37□ |
| | nasopharyngeal carcinoma cells | | |
| CNE2 | Human lowly differentiated | adhesive | 8% FBS-RPMI1640, 5% CO₂, 37□ |
| | nasopharyngeal carcinoma cells | | |
| A549 | Human non-small cell lung cancer | adhesive | 6% FBS-RPMI1640, 5% CO₂, 37□ |
| | cell line | | |
| PC-9 | human cell lung cancer cell line | adhesive | 10% FBS-RPMI1640, 5% CO₂, 37□ |
| MKN-45 | human gastric cancer cell line | adhesive | 10% CS-RPMI1640, 5% CO₂, 37□ |

Determination of Inhibition Rate to Seven Types of Cancer Cell Lines by MTT Method

Test solutions of Compound 5 prepared as aforementioned were used to treat the seven types of cancer cell lines for 72 hs. 104, 3-(4,5-dimethylthiazol-2-yl)-2,5-diphenyltetrazolium bromide (MTT) (5 mg/ml) were added to each well and continued cultivation for 4 hs. After removal of the solutions in the wells, 2000 μL DMSO was added, the plates were well shook for complete dissolve. OD was measured with microplate reader under wavelength of 492 nm and then the inhibition rate was calculated. The inhibitory effect of each sample solution on each cancer cell line was assayed three times in parallel.

Software program Origin 7.5 was used for analyzing the OD data obtained in the testing wells. IC50 to each human cancer cell lines are calculated and shown in the following Table 3.

TABLE 3
Inhibition effects of Compound 5 on human cancer cell lines

| Cell line | IC50 (μg/mL) |
|---|---|
| A549 | 13.2 ± 0.91 |
| PC-9 | 21.8 ± 1.3 |
| K562 | 16.8 ± 2.8 |
| HL60 | 18.3 ± 3.0 |
| CNE1 | 49.29 ± 2.8 |
| CNE2 | 13.3 ± 2.6 |
| MKN-45 | 33.2 ± 3.1 |

Compound 5 had shown significant inhibitory effect on all the seven types of human cancer cell lines. IC50 was in the range of 13.2±0.91 to 49.29±2.8 μg/mL. It shows Compound 5 can inhibit a broad scope of cancers, among which, Compound 5 had the best inhibitory effect on A549.

### Example 3 In Vivo Anti-Tumor Activity Evaluation of Compound 5 on Animal Model

Compound 5 obtained in Example 1 was subjected to the in vitro anti-tumor activity evaluation.

1. Cell Line and Laboratory Animal

Human non-small cell lung cancer cell line (A549) was purchased from Shanghai Chinese Academy of Sciences Cell Bank and stored in School of Medicine of Fujian Medical University.

3-4-week-old nude BALB-c mice weighted 18±2 g, purchased from SLRC laboratory Animal Co. Ltd., Shanghai, China ( [symbol] [symbol] [symbol] ), Laboratory Animal License: SOCK ( [symbol] ) 2012-0002, Animal License: 2007000537438).

The nude mice were fed in the SPF-system, six mice in a cage. The nude mice were provided with standard food and water, maintained in the light and dark cycling conditions (12/12 h), room temperature 25±2° C., humidity 50±10%. The water and beddings were steriled and changed every day. The mice were fed in the maintenance room for a week before the experiments.

2. Preparation of Test Solution of Compound 5

Compound 5 was poorly dissolved in water. Its water solution (or saline solution) was acidic. When the pH of the solution was adjusted to be with the injection acceptable range (6.0-8.0), the solubility was enhanced a little, but it was still quite low, thus led to precipitation. Suitable co-solvents were selected to enhance the solubility. Studied the solubility enhancing effects of Tween-80 (final concentration 0.02-2%, v/v), PEG-400 (final concentration <40%, v/v), 1, 2-propanediol (final concentration <40%, v/v), glycerol (final concentration <30%, v/v), and CMC-Na (final concentration 1-5%, w/v). It was found that, within the injection acceptable range, Tween-80 and PEG-400 showed a better solubility enhancing effect. Considering formulations comprising tween compounds may lead to hemolysis, PEG-400 was selected as the co-solvent. After comparing solubility enhancing effects of different ratio of PEG-400, it was found that 10% PEG-400 had the best solubility enhancing effect.

Accurately weighed 6 mg of compound 5 powder, added cosolvent PEG-400 (100 μL), vortex and treated with ultrasonic. After being fully dissolved, a small amount of physiological saline was gradually added into the solution, adjusted the pH to around 7.4, and made the solution to 1 mL.

3. Preparation of Positive Control Solution

Accurately weighed a certain amount of injection grade doxorubicin powder, added saline to obtain a 0.4 mg/mL test solution. The test solution is prepared avoiding light and handy for use.

4. Construction of Human Tumor Cell A549 Allograft Nude Mouse Model

4.1 Cell Culture

Human non-small cell lung cancer cell line A549 was resuscitated. The ratio of FBS in the culture was increased from 6% to 10%, so as to make the cells proliferated rapidly. When the cell coverage in the culture flask reached 80% or so (i.e., logarithmic growth phase), the A549 cells were digested and suspended. Using autoclaved PBS to wash the cells twice to remove the residual culture medium. And then the cells were resuspended with PBS and adjusted the cells concentration to 1×10⁷ cells/mL.

4.2 Construction of Nude Mouse Model

After the mice accustomed to the environment, under steriled condition, using disposable syringe to transfer A549 cells (1×10⁷/mL) and subcutaneously inoculated in the flank of nude mice. 0.2 ml of suspension of A549 cells was injected into each nude mice and the procedure was finished within 30 min. The rest of the cells were subjected to culture and observed for any contamination, so as to provide information on whether the construction of animal model was success.

4.3 Grouping of the Testing Animals

After the inoculation of cancer cells was success and the volume of xenograft tumors reached 50 to 100 mm³, the animals were divided randomly into 5 groups (high dosage, middle dosage, low dosage, positive control, blank control) and each group comprised 6 mice.

5. Study on In Vivo Anti-Tumor Effect of Compound 5

5.1 Animal Grouping

According to the animal grouping, compound 5 testing solutions were injected via tail vein, the corresponding injection dosage were: high dosage 30 mg/kg, middle dosage 15 mg/kg, low dosage 5 mg/kg. The animals were administered once per day. The injection volume was adjusted according to the weight measured. The animals were administered for 12 days. Positive control group was injected with 2 mg/kg Doxorubicin. The animals were administered once every 3 days, and 4 times in the 12-day period. Blank control group was injected with saline solution.

5.2 Measurement of Tumor Size

During the administration period, the living and mental state of the mice were observed every day. Body weight and tumor size was measured and recorded. The tumor size was measured using Vernier caliper and tumor volumes were calculated using the formula: V (mm³)=½×L (mm)×W² (mm²) in which L is the length and W is the width of the solid tumor nodules.

24 hrs after the last administration, the nude mice were sacrificed and the tumor was removed for volume measurements. Tumor inhibition rate was calculated. Tumor inhibition rate (%)=(1−tumor weight of testing group/tumor weight of blank control group)×100%.

5.3 Treatment of Mice During Administration Period

Each mouse was weighted 24 hours after administration. In the end of the experiment, the mice were sacrifice by treating at the neck area. The tumor was removed for volume measurements and took photos.

Animals were handled according to Regulations on the administration of laboratory animals (Order No. second of the State Science and Technology Commission of the People's Republic of China, No. 1988).

5.4 Statistical Analysis

The experimental data were analyzed by SPSS 13.0 software Comparison between groups were carried out by one-way ANOVA or two-sample comparison of U-test. Results were provided in mean±SE, difference with P<0.05 was deemed significant.

During the 12 days of continuous treatment process on the 5 groups of animals, the nude mice food intake were normal and no discomfort were observed, There was a slight increase in body weight on the mice. The growth rate of the tumor in the control group was significantly faster than that in the other groups. The inhibition of the tumor growth was obvious in the four groups of nude mice treating with drugs, and no obvious side effects were observed. The tumor volume change curve of the nude mice was plotted according to the tumor volume calculated from the daily tumor size.

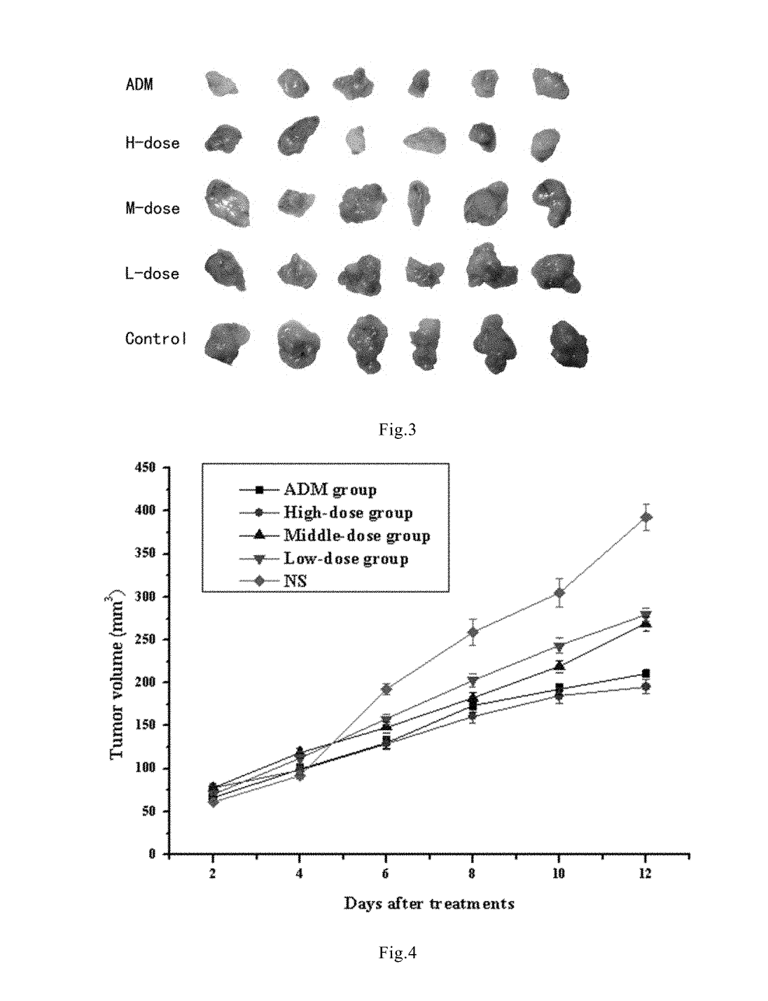

As can been seem from FIG. 3, the separated ventral subcutaneous transplanted tumor specimens were a single tumor mass and had no adhesion to the skin. The out appearance was red and white and had an irregular round or oval shape. The membrane of the tumors were intact. The surface had rich vascular distribution. There were no basal adhesion to the ventral muscles. The tissue texture were tough and hard, and their cutting surface was gray. There were no obvious local infiltration.

The average tumor inhibition rate after drug treatment was calculated and the results were as below: Positive control group (Doxorubicin) 56.8±5.6%, Compound 5 high dosage group 62.2±21.3%, Compound 5 middle dosage group 43.7±19.8%, Compound 5 low dosage group 36.9±6.1%. The results were shown in Table 4 below.

TABLE 4
Drug dosage, weights of before and after treatment, tumor weights and tumor
weight-inhibitions of e in vivo experiments of Compound 5, (n = 6)

| | | Weights (g) | | | |
|---|---|---|---|---|---|
| Animal | Dosage | Before | After | Tumor | Tumor Weight- |
| Groups | (mg/kg) | treatment | treatment | weights (g) | inhibitions(%) |
| Blank Conrtol | NS + 10% PEG-400 | 23.7 ± 1.1 | 25.4 ± 1.2 | 0.471 ± 0.092 | — |
| Doxorubicin | 2 mg/kg | 22.7 ± 1.9 | 23.6 ± 1.1 | 0.220 ± 0.037 | 56.8 ± 5.6 |
| High | 30 mg/kg | 22.4 ± 1.2 | 24.4 ± 1.6 | 0.193 ± 0.056 | 62.2 ± 21.3 |
| Middle | 15 mg/kg | 23.9 ± 1.5 | 24.9 ± 1.0 | 0.287 ± 0.101 | 43.7 ± 19.8 |
| Low | 5 mg/kg | 22.8 ± 0.8 | 23.4 ± 0.7 | 0.305 ± 0.039 | 36.9 ± 6.1 |

The change of the size of tumor volume in nude mice under continuous administration was shown in FIG. 3 and FIG. 4. There was significant difference between the different dosage of Compound 5 treatment groups, as well as between the treatment group and the positive control group and the negative control group (P<0.05).

In the allograft nude mouse model, Compound 5 could significantly inhibit the proliferation of A549 cells xenograft and reduce the tumor growth rate. The high dosage (30 mg/kg) of Compound 5 had a better inhibitory effect than the positive control drug doxorubicin. Doxorubicin is one of the clinically commonly used drugs for lung cancer. In the allograft nude mouse model, the middle dosage of compound 5 (15 mg/kg) and low dosage (5 mg/kg) also showed good inhibition of tumor cell proliferation effect. The tumor volume of the high dosage group was obviously smaller than that of the low-dosage group, indicating a dose-dependent effect.

Therefore, it can be seem that Compound 5 could effectively inhibit the growth of human non-small cell lung cancer cells A549 solid tumor in nude mice. The toxicity thereof was low and nude mice were well tolerated to the drugs. Therefore, Compound 5 is a potential highly efficient, low toxicity of anti-tumor active ingredient.

The compounds provided by the present invention showed significant anti-tumor effects on multiple human cancer cell lines, including lung cancer (such as non-small cell lung cancer and small cell lung cancer), stomach cancer, leukemia (Including acute promyelocytic leukemia and chronic myeloid leukemia) and esophageal cancer.

These compounds also showed promising anti-tumor effects in the animal bodies. The experimental results showed that the compounds of the present invention are effective in inhibiting various cancer cells, particularly lung cancers, such as non-small cell lung cancer in the animal bodies.

The above description of the present invention can not be construed as limiting the present invention. Unless otherwise indicated, the practice of the present invention will use conventional techniques such as organic chemistry, polymer chemistry, biotechnology, etc. And it is obviously that the invention may be practiced otherwise than as specifically described in the foregoing description and examples. Other aspects and modifications within the scope of the invention will be apparent to those skilled in the art to which the invention pertains. Many changes and variations are possible in accordance with the teachings of the present invention and are therefore within the scope of the invention.

Unless otherwise indicated, the unit of temperature, i.e., degree as present herein refers to Celsius degree, i.e., ° C.

1. (canceled)

2. (canceled)

3. A pharmaceutical composition for treating cancer, which comprises a compound of Formula I or a pharmaceutically acceptable salt thereof and a pharmaceutically acceptable carrier: 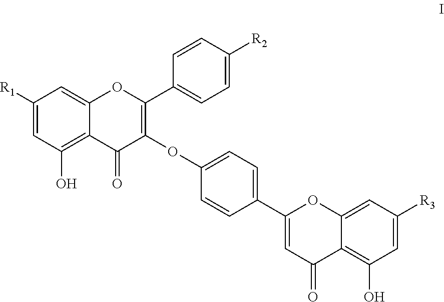
wherein R₁, R₁ and R₃ are each independently selected from H, hydroxy, lower alkyl, lower alkenyl, lower alkoxy, halo, amino, hydroxyalkyl, aminoalkyl, nitro, aryl and heteroaryl, and wherein the pharmaceutical composition is sterile.

4. The pharmaceutical composition according to claim 3, wherein said cancer is selected from skin cancer, lung cancer, Kaposi's sarcoma, testicular cancer, lymphoma, leukemia, esophageal cancer, stomach cancer, colon cancer, breast cancer, endometrial cancer, ovarian cancer, central nervous system cancer, liver cancer and prostate cancer.

5. The pharmaceutical composition according to claim 4, wherein said cancer is lung cancer.

6. A method for treating cancer, which comprises administering to a subject in need thereof a treatment effective amount of a compound of Formula I or a pharmaceutically acceptable salt thereof: 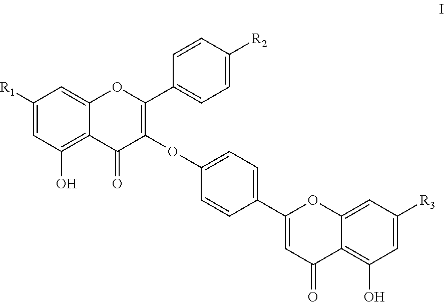
wherein R₁, R₁ and R₃ are each independently selected from H, hydroxy, lower alkyl, lower alkenyl, lower alkoxy, halo, amino, hydroxyalkyl, aminoalkyl, nitro, aryl and heteroaryl.

7. The method according to claim 6, wherein said cancer is selected from skin cancer, lung cancer, Kaposi's sarcoma, testicular cancer, lymphoma, leukemia, esophageal cancer, stomach cancer, colon cancer, breast cancer, endometrial cancer, ovarian cancer, central nervous system cancer, liver cancer and prostate cancer.

8. The method according to claim 7, wherein said cancer is lung cancer.

9. (canceled)

10. (canceled)

11. (canceled)

12. (canceled)

13. (canceled)

14. The pharmaceutical composition according to claim 3, wherein the pharmaceutical composition is formulated for intravenous administration.

15. The pharmaceutical composition according to claim 3, which comprises a compound 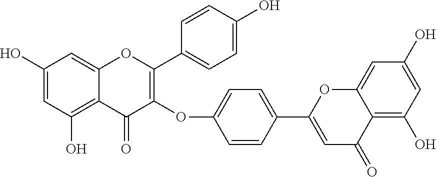 [SMILES: O=c1cc(-c2ccc(Oc3c(-c4ccc(O)cc4)oc4cc(O)cc(O)c4c3=O)cc2)oc2cc(O)cc(O)c12]
or a pharmaceutically acceptable salt thereof and a pharmaceutically acceptable carrier.

16. The pharmaceutical composition according to claim 5, wherein said cancer is non-small cell lung cancer.

17. The method according to claim 6, which comprises administering to a subject in need thereof a treatment effective amount of a compound 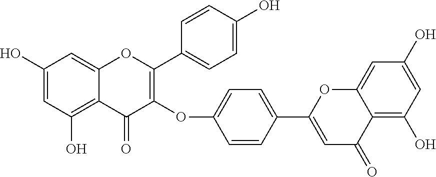 [SMILES: O=c1cc(-c2ccc(Oc3c(-c4ccc(O)cc4)oc4cc(O)cc(O)c4c3=O)cc2)oc2cc(O)cc(O)c12]
or a pharmaceutically acceptable salt thereof.

18. The method according to claim 8, wherein said cancer is non-small cell lung cancer.
